(19) United States
(12) Patent Application Publication
(10) Pub. No.: US 2021/0368809 A1
(43) Pub. Date: Dec. 2, 2021

(54) Nutritional Cereal-based Food with Low Glycemic Load and Processing Method thereof

(71) Applicant: Jiangnan University, Wuxi (CN)

(72) Inventors: Ming MIAO, Wuxi (CN); Xiao ZHOU, Wuxi (CN); Yungao LI, Wuxi (CN); Yuqi YANG, Wuxi (CN); Tao ZHANG, Wuxi (CN); Zhengyu JIN, Wuxi (CN); Zhiyuan LIU, Wuxi (CN)

(21) Appl. No.: 17/403,932

(22) Filed: Aug. 17, 2021

(30) Foreign Application Priority Data
Oct. 16, 2020 (CN) 202011108990.2

(51) Int. Cl. A21D 2/36; A23L 7/109; A21D 13/047; A21D 10/04; A23L 33/185; A23L 33/105

(52) U.S. Cl. CPC A21D 2/36; A23L 7/109; A23L 33/105; A21D 10/04; A23L 33/185; A21D 13/047

(57) ABSTRACT

The disclosure discloses a nutritional cereal-based food with low glycemic load and processing method thereof, and belongs to the technical field of processing of healthy foods. According to the disclosure, bulk grain crops are used as raw materials and mixed with a natural plant extract with the content of 3-deoxyanthocyanidin more than 3 mg/100 g and a sulfur-rich plant or animal protein, so that in-situ encapsulation of starch granules is achieved through an interaction of a sulfur-containing protein and 3-deoxyanthocyanidin, and a grain-source nutritional food for special dietary uses with low GI. According to the method of the disclosure, not only is the process environmentally friendly, operation procedures are simple, but also slow release of blood glucose after a meal is achieved by using the product, and the product can be used as a meal replacement food for people with abnormal glucose metabolism and the like.

### TECHNICAL FIELD

The disclosure relates to a nutritional cereal-based food with low glycemic load and processing method thereof, and belongs to the technical field of processing of healthy foods.

### BACKGROUND

With the development of economy in China, people's living standards are improved significantly, and people's demands for food safety and nutrition are increased urgently. At the same time, patients suffering from non-communicable chronic diseases and metabolic syndromes related to dietary structures and living habits are increased sharply. According to surveys, at present, the number of obese and overweight people in China is nearly 300 million, the number of people with impaired glucose tolerance is about 150 million, and the number of diabetics is higher than 90 million. A rapid rise trend of these chronic diseases and risk factors thereof has become a prominent problem affecting the health of people in China. The World Health Organization (WHO)'s assessment of the factors affecting human health shows that the effect of dietary nutrition factors (13%) on health is only lower to that of genetic factors (15%) and higher than that of medical factors (8%). Therefore, under the premise of ensuring food safety, dietary intervention and nutritional regulation have become the most urgent link to be improved in order to increase the health level and ensure and improve people's livelihood, and daily diet intervention is a key part.

As typical daily staple foods of Chinese residents and main sources of energy intake for the body, grains account for 55%-75% of total energy intake, and regulation of blood glucose metabolism and energy balance after a meal is directly affected by “quality” and “quantity”. At present, some sustained-release energy foods sold on the market achieve functions mainly by adding exogenous processed substances such as isomaltulose, arabinose, pullulan, acarbose and a white kidney bean extract, and the catalytic activity of digestive enzymes is partially inhibited by these products, so that the digestion and degradation rates of starch are reduced, and the phenomenon of a hypoglycemia peak occurs after a meal, however, clinical trials prove that metabolic syndromes such as insulin resistance and energy balance disorders are likely to be caused when large amounts of products mentioned above are eaten. Therefore, in order to solve the problems above, it is urgent to find a processing method of a grain-source nutritional food for special dietary uses with a high nutritional value and a low glycemic load (GL).

### SUMMARY

An objective of the disclosure is to provide a processing method of a grain-source nutritional food for special dietary uses with a high nutritional value, a good health care function and a low glycemic load. The processing method of the disclosure has the characteristics of simple process, advanced technology, high safety, easy process control, continuous production and the like, and industrialized production is easy.

Specifically, the disclosure first provides a processing method of a nutritional cereal-based food with low glycemic load, and the method includes the following steps:

(1) weighing a certain mass of natural plant tissue, pulverizing the natural plant tissue, adding an acetic acid solution with a concentration of 1-5 wt % according to a weight ratio of 1:3 to 1:10, placing the mixture in a water bath for extraction treatment at 30-50° C. for 5-120 minutes, and then collecting a supernatant to obtain a natural plant extract;

(2) pulverizing a grain crop to 100-120 mesh, uniformly mixing 70-100 parts by mass of the grain crop powder, 0-30 parts by mass of a protein, the natural plant extract accounting for 0.01-3 wt % of the total mass of the grain crop powder and the protein and an appropriate amount of water to prepare dough or batter, and then preparing a nutritional cereal-based food with low glycemic load by using a general food production process of rice and noodles.

In an embodiment of the disclosure, the content of protein in the dough or batter is no less than 10 parts.

In an embodiment of the disclosure, the natural plant tissue is any one or more of flowers, leaves and stalks of plants such as sorghum, sugarcane, corn, camellia japonica, gesneriaceae, grapes, quinoa, highland barley, mosses and ferns.

In an embodiment of the disclosure, the content of 3-deoxyanthocyanidin in the natural plant extract is more than 3 mg/100 g.

In an embodiment of the disclosure, the grain crop is any one or more of rice, wheat, barley, oats, corn, foxtail millet, sorghum, milled foxtail millet, hulless oats, broom corn millet, mixed beans and potatoes.

In an embodiment of the disclosure, the protein is one or more of sulfur-rich plant or animal proteins, and the plant or animal proteins are proteins derived from plants or animals, preferably one or more of ovalbumin, glutenin, soy protein isolate or lactoglobulin.

In an embodiment of the disclosure, the general food production process of rice and noodles includes, but is not limited to, production processes of noodles, steamed buns, steamed stuffed buns, fried foods and baked foods, and production processes in Principles of Cereal Science and Technology published by American Association of Cereal Chemists International can also be taken as references.

In an embodiment of the disclosure, the appropriate amount of water is a conventional amount used by those skilled in the art for preparing rice and noodles, preferably 30-90 parts.

Second, the disclosure also provides a nutritional cereal-based food with low glycemic load prepared by using the above method.

In an embodiment of the disclosure, the nutritional cereal-based food with low glycemic load has a GI of lower than 55, and a GL of lower than 10.

Third, the disclosure also provides a food containing the above nutritional cereal-based food with low glycemic load.

In an embodiment of the disclosure, the food includes, but is not limited to, food for a special medical purpose, a specific food for athletes and a nutritional food, and the content of slowly digestible starch is more than 50%.

At last, the disclosure provides application of the above nutritional cereal-based food with low glycemic load in the fields of food and medicine.

The disclosure has the following advantages:

(1) In the disclosure, grain crops commonly sold in the domestic market are used as raw materials which have a wide source without limitation to production place and season, and industrialized production is easy.

(2) In the disclosure, a food-based component packaging technology is adopted, namely, in-situ encapsulation is achieved through an interaction of a sulfur-containing protein and 3-deoxyanthocyanidin so that GL of a final product can be significantly reduced, and a high nutritional value of the product is ensured; at the same time, a clean production process is adopted in the disclosure, so that the environment is basically not polluted.

(3) In the disclosure, by using the technology, continuous production can be achieved, procedures are simple, and automatic control is easy.

(4) The product of the disclosure has the functions of preventing and treating diseases and maintaining health of the human body. Not only are standard requirements of the food for a special medical purpose for diabetics (GI is less than 55, and the general rule of formula foods for special medical purposes GB 29922-2013 is taken as a reference), but also the food can be used as a special energy slow-release product for athletes or an ideal nutritional food for healthy people (the content of slowly digestible starch is higher than 40% and is a health effect value stipulated by EU Food Safety Agency (EFSA)), the market prospect is broad, and economic and social benefits are high.

### DETAILED DESCRIPTION

Measurement of glycemic index (GI) and glycemic load (GL): GI and GL are calculated based on an increase in an area under a postprandial glucose response curve of a person eating 50 g of food, the glycemic concentration is determined by using a GOD-POD colorimetric method, and the glycemic load (GL) is a product of the mass of available carbohydrates in the food and a GI value.

A measurement method of 3-deoxyanthocyanidin: An Agilent 1200 HPLC system is adopted for analysis, a chromatographic column ZORBAX XDB which is 5 μm and 4.6*250 mm is used. A mobile phase A includes 1% formic acid, and a mobile phase B includes 80% methanol and 1% formic acid. Gradient elution involving 100% of A for 0-5 minutes and 100% of A to 20% of A and 80% of B for 5-50 minutes is adopted. The flow rate is 1 mL/min, the column temperature is 30° C., and the injection volume is 20 μL.

Measurement of the content of slowly digestible starch: An Englyst method is used, a to-be-tested sample and a mixed enzyme solution (a-amylase and glucoamylase) are subjected to an oscillation reaction at 160 r/min in a water bath at 37° C. for 20 minutes and 120 minutes under the condition of simulating in-vitro intestinal digestion (pH 5.2), 0.5 ml of a supernatant is taken, and the content of glucose is measured by using a GOD-POD colorimetric method. The content of slowly digestible starch is (G120-G20)*0.9/W, where G20 is the amount of glucose produced after hydrolysis for 20 minutes (mg); G120 is the amount of glucose produced after hydrolysis for 120 minutes (mg); W is the amount of the to-be-tested sample (mg).

The following examples are used to further illustrate the disclosure, but not to limit the embodiments of the disclosure.

### EXAMPLE 1

5 g of camellia leaves were weighed and pulverized. An acetic acid solution with a mass percentage concentration of 1% was added according to a weight ratio of 1:3. The mixture was placed in a water bath at 30° C. for extraction treatment for 60 minutes. Then a supernatant was collected (after measurement, the content of 3-deoxyanthocyanidin was 3.2 mg/100 g). Wheat was mechanically milled and pulverized to 120 mesh. 90 parts by mass of wheat flour, 10 parts by mass of soy protein isolate and a camellia leaf extract accounting for 1% of the total mass of the wheat flour and the protein were uniformly mixed. 55 parts by mass of water was added and stirred to prepare wheat dough Then a nutritional food for special dietary uses was prepared by using a food production process of noodles.

After measurement, the prepared noodles had a GI of 51 and a GL of 8.9, and the content of slowly digestible starch was 54%. Therefore, the nutritional food was a nutritional cereal-based food with low glycemic load.

### EXAMPLE 2

10 g of sorghum seedlings were weighed and pulverized. An acetic acid solution with a mass percentage concentration of 1% was added according to a weight ratio of 1:10. The mixture was placed in a water bath at 30° C. for extraction treatment for 15 minutes. Then a supernatant was collected (after measurement, the content of 3-deoxyanthocyanidin was 5.0 mg/100 g). Rice was mechanically milled and pulverized to 100 mesh. 70 parts by mass of rice flour, 30 parts by mass of ovalbumin and a sorghum seedling extract accounting for 3% of the total mass of the rice flour and the protein were uniformly mixed. 45 parts by mass of water was added and stirred to prepare rice dough. Then a nutritional food for special dietary uses was prepared by using a food production process of baked foods.

After measurement, the prepared noodles had a GI of 48 and a GL of 9.4, and the content of slowly digestible starch was 65%. Therefore, the nutritional food was a nutritional cereal-based food with low glycemic load.

### EXAMPLE 3

10 g of sugarcane stalks were weighed and pulverized. An acetic acid solution with a mass percentage concentration of 3% was added according to a weight ratio of 1:8. The mixture was placed in a water bath at 40° C. for extraction treatment for 100 minutes. Then a supernatant was collected (after measurement, the content of 3-deoxyanthocyanidin was 3.1 mg/100 g). Corn was mechanically milled and pulverized to 110 mesh. 90 parts by mass of corn flour, 10 parts by mass of glutenin and a sugarcane stalk extract accounting for 2% of the total mass of the corn flour and the protein were uniformly mixed. 38 parts by mass of water was added and stirred to prepare corn dough. Then a nutritional food for special dietary uses was prepared by using a food production process of steamed buns.

After measurement, the prepared noodles had a GI of 52 and a GL of 9.0, and the content of slowly digestible starch was 52%. Therefore, the nutritional food was a nutritional cereal-based food with low glycemic load.

When any one or more of flowers, leaves and stalks of sorghum, sugarcane, corn, camellia japonica, gesneriaceae, grapes, quinoa, highland barley, mosses and ferns is used as a natural plant extract, any one or more of rice, wheat, barley, oats, corn, foxtail millet, sorghum, milled foxtail millet, hulless oats, broom corn millet, mixed beans and potatoes is used as a starch source, and a nutritional cereal-based food with low glycemic load with GI lower than 55, GL lower than 10 and the content of slowly digestible starch higher than 50% can be prepared by using methods in Examples 1 to 3.

Comparative Example 1

5 g of camellia leaves were weighed and pulverized. An acetic acid solution with a mass percentage concentration of 1% was added according to a weight ratio of 1:3. The mixture was placed in a water bath at 30° C. for extraction treatment for 60 minutes. Then a supernatant was collected (after measurement, the content of 3-deoxyanthocyanidin was 3.2 mg/100 g). Wheat was mechanically milled and pulverized to 120 mesh. 90 parts by mass of wheat flour and a camellia leaf extract accounting for 1% of the mass of the wheat flour were uniformly mixed. 40 parts by mass of water was added and stirred to prepare wheat dough. Then noodles were prepared by using a food production process of noodles.

After measurement, the prepared noodles had a GI of 62 and a GL of 14.3, and the content of slowly digestible starch was 34%.

Comparative Example 2

Wheat was mechanically milled and pulverized to 120 mesh. 90 parts by mass of wheat flour and 10 parts by mass of soy protein isolate were uniformly mixed. 55 parts by mass of water was added and stirred to prepare wheat dough. Then noodles were prepared by using a food production process of noodles.

After measurement, the prepared noodles had a GI of 59 and a GL of 13.8, and the content of slowly digestible starch was 32%.

### Comparative Example 3

1 g of camellia leaves were weighed and pulverized. An acetic acid solution with a mass percentage concentration of 1% was added according to a weight ratio of 1:15. The mixture was placed in a water bath at 30° C. for extraction treatment for 60 minutes. Then a supernatant was collected (after measurement, the content of 3-deoxyanthocyanidin was 1.7 mg/100 g). Wheat was mechanically milled and pulverized to 120 mesh. 90 parts by mass of wheat flour and a camellia leaf extract accounting for 1% of the mass of the wheat flour were uniformly mixed. 55 parts by mass of water was added and stirred to prepare wheat dough. Then noodles were prepared by using a food production process of noodles.

After measurement, the prepared noodles had a GI of 58 and a GL of 14.2, and the content of slowly digestible starch was 34%.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A method of processing a nutritional cereal-based food with low glycemic load, comprising the following steps:
(1) weighing a predetermined mass of natural plant tissue, pulverizing the natural plant tissue, adding an acetic acid solution with a concentration of 1-5 wt % according to a weight ratio of 1:3 to 1:10, placing the mixture for extraction treatment at 30-50° C. for 5-120 minutes, and then collecting a supernatant to obtain a natural plant extract;
(2) pulverizing a grain crop to 100-120 mesh, uniformly mixing 70-100 parts by mass of the grain crop powder, 0-30 parts by mass of a protein, the natural plant extract accounting for 0.01-3 wt % of the total mass of the grain crop powder and the protein and an predetermined amount of water to prepare dough or batter, and then preparing the nutritional cereal-based food with low glycemic load by using a general food production process of rice and noodles.

2. The method of claim 1, wherein the natural plant tissue is any one or more of flowers, leaves and stalks of sorghum, sugarcane, corn, camellia japonica, gesneriaceae, grapes, quinoa, highland barley, mosses and ferns.

3. The method of claim 1, wherein content of 3-deoxyanthocyanidin in the natural plant extract is more than 3 mg/100 g.

4. The method of claim 1, wherein the grain crop is any one or more of rice, wheat, barley, oats, corn, foxtail millet, sorghum, milled foxtail millet, hulless oats, broom corn millet, mixed beans and potatoes.

5. The method of claim 1, wherein the protein is one or more of sulfur-rich plant or animal derived proteins.

6. The method of claim 5, wherein the protein comprises one or more of ovalbumin, glutenin, soy protein isolate and lactoglobulin.

7. The method of claim 6, wherein the content of the protein in the dough or batter is no fewer than 10 parts.

8. The method of claim 1, wherein the general food production process of rice and noodles comprises production processes of noodles, steamed buns, steamed stuffed buns, fried foods and baked foods.

9. The method of claim 1, wherein the predetermined amount of water is 30-90 parts.

10. The nutritional cereal-based food with low glycemic load prepared by the method of claim 1.

11. The nutritional cereal-based food with low glycemic load of claim 10, wherein the nutritional cereal-based food with low glycemic load has a glycemic index (GI) of less than 55, and a glycemic load (GL) of less than 10.

12. A product comprising the nutritional cereal-based food with low glycemic load according to claim 10.

13. The product of claim 12, wherein the product has a medical use, sports or athletic training related use, or a use in nutritional supplement.
